(19) United States
(12) Patent Application Publication
(10) Pub. No.: US 2009/0012120 A1
(43) Pub. Date: Jan. 8, 2009

(54) Synthesis of N-heterocycles, beta-amino acids, and allyl amines via aza-payne mediated reaction of ylides and hydroxy aziridines

(75) Inventors: Babak Borhan, East Lansing, MI (US); Jennifer M. Schomaker, Berkeley, CA (US); Somnath Bhattacharjee, East Lansing, MI (US); Keith Korthals, East Lansing, MI (US)

Correspondence Address: Ian C. McLeod;IAN C. MCLEOD, P.C., 2190 Commons Parkway, Okemos, MI, 48864, US

(73) Assignee: Board of Trustees of Michigan State University, East Lansing, MI (US)

(21) Appl. No.: 11/801,395

(22) Filed: May 9, 2007

Related U.S. Application Data
(60) Provisional application No. 60/799,086, filed on May 10, 2006.

(51) Int. Cl. A61K 31/4015; C07D 207/12; C07D 209/32; C07D 209/54; C07C 211/00; C07D 211/00; A61P 35/00; A61K 31/13; A61K 31/403; A61K 31/445

(52) U.S. Cl. 514/315; 548/556; 548/541; 548/512; 548/408; 564/463; 546/184; 514/579; 514/409; 514/412; 514/424

(57) ABSTRACT

An ylide-based aza-Payne rearrangement of 2,3-aziridin-1-ols leads to an efficient process for the preparation of pyrrolidines. The aza-Payne rearrangement under the basic reaction conditions favors the formation of epoxy amines. Subsequent nucleophilic attack of the epoxide by the ylide yields a bis-anion, which upon a 5-exo-tet ring closure yields the desired pyrrolidine, thus completing the relay of the 3-membered the 5-membered nitrogen containing ring system. This process takes place with complete transfer of stereochemical fidelity, and can be applied to sterically hindered aziridinols.

### CROSS-REFERENCE TO RELATED APPLICATIONS

This application claims benefit to U.S. Provisional Application Ser. No. 60/799,086, filed May 10, 2006, which is incorporated herein by reference in its entirety.

### STATEMENT REGARDING FEDERALLY SPONSORED RESEARCH OR DEVELOPMENT

Not Applicable.

### BACKGROUND OF THE INVENTION

(1) Field of the Invention

The present invention relates to a process for the preparation of pyrrolidines using a sulfoxonium ylide by a one-carbon homologative relay ring expansion. The pyrrolidines can be diastereomerically and enantiomerically pure depending upon the starting epoxy amine. The compounds are intermediate to pharmaceutical compounds, particularly in chiral form.

(2) Description of Related Art

Substituted pyrrolidines are important heterocycles by virtue of their frequent appearance in a large number of biologically active natural products and pharmaceuticals. Enantiomerically pure pyrrolidines are also used as chiral auxiliaries for various organic transformations.² Conformationally restrained analogues of proline are also being utilized in the synthesis of unnatural oligomers as scaffolds for biological applications such as antimicrobial activity.³ As such, much effort has been devoted to the synthesis of pyrrolidines in enantiomerically and diastereomerically pure form, including, but certainly not limited to, 3+2 cycloadditions of azomethine ylides with alkenes or nitrones with cyclopropanes, 4 oxidative decarboxylation-β-iodination of amino acids,⁵ palladium-catalyzed carboamination reactions,⁶ intramolecular cyclization of epoxy and halogenated sulfones under basic conditions,⁷ acid-catalyzed cyclization of vinylsilanes,⁸ intramolecular carbolithiation of homoallylic amines,⁹ radical cyclizations,¹⁰ Brønsted acid-catalyzed intramolecular hydroamination of alkenylamines,¹¹ manipulations of sugars from the chiral pool,¹² various other metal-catalyzed cyclizations,¹³ and ring-closing metathesis.¹⁴ Clearly, the importance of pyrrolidines can be directly implied from the significant amount of effort that has led to the development of various methodologies for their synthesis.

The Payne rearrangement is a base-mediated isomerization of epoxy alcohols and has been well-utilized in organic synthesis to reveal the latent electrophilicity at C-1 of a 2,3-epoxy-1-ol such as 1 (Scheme 1) 15 We have recently used this approach to control attack at C-1 with dimethylsulfoxonium methylide in the synthesis of a series of 2,3-substituted tetrahydrofurans (Scheme 1).^{16a} These reactions can be high-yielding and deliver the THF ring in a regio- and stereocontrolled manner. The Payne rearrangement of trans epoxides is not as facile as cis epoxides (release of steric strain of cis epoxides is the driving force) as can be seen from comparing yields of THF products 3 and 6, which originate from cis and trans epoxy diols 1 and 4, respectively (Scheme 1). However, the presence of an electron-withdrawing atom at C-4 or C-5 of the epoxy alcohol is sufficient for successful THF formation with 2,3-disubstituted epoxy-1-ols. Certain substrates, mainly alkyl-disubstituted and trisubstituted 2,3-epoxy-1-ols,

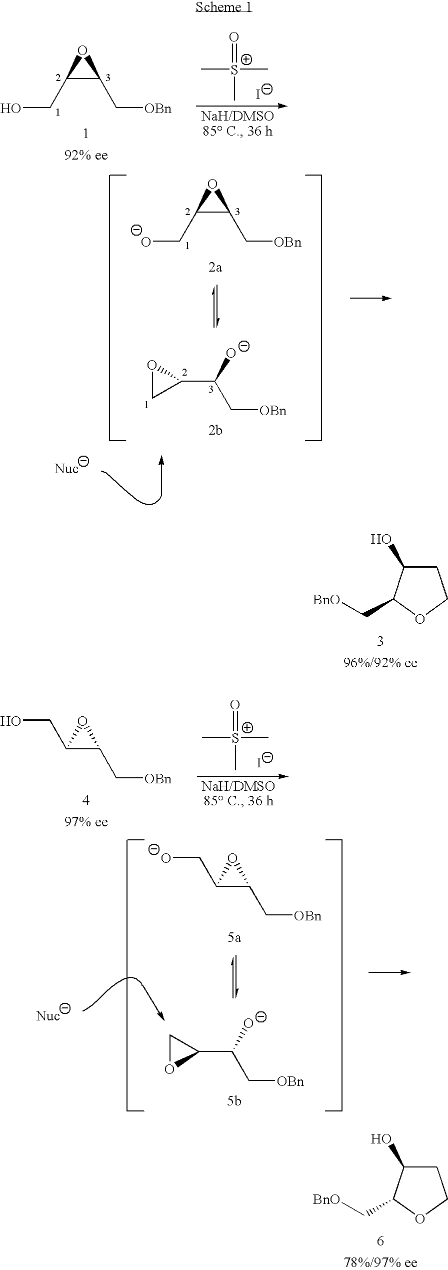

do not undergo sufficient Payne rearrangement to allow for successful nucleophilic attack on the less hindered 1,2-epoxy-3-ol (structures analogous to 2b and 5b in Scheme 1). Mixtures of products often result from competing nucleophilic attack at C-2 and C-3, as well as base-mediated elimination reactions.

There is a need for a process to which allows the preparation of pyrrolidines.

### OBJECTS

It is therefore an object of the present invention to provide a process for the preparation of di or tri substituted pyrrolidines, preferably in a one-pot reaction from a 2,3-aziridin-1-ol through a 1,2-epoxy-3-ol protected amine using dimethylsulfoxonium methylide. It further is an object of the present invention to provide a process which produces the pyrrolidines in high yield. These and other objects will become increasingly apparent by reference to the following description and the drawings.

### SUMMARY OF THE INVENTION

The present invention relates to a process for the preparation of a 2,3-di- or tri-substituted pyrrolidine which comprises reacting 1,2-epoxy-3-N protected amine with dimethylsulfoxonium methylide in an aprotic solvent to produce the 2,3-di- or tri-substituted pyrrolidine. Preferably, the 1,2-epoxy-3-protected amine and the 2,3-pyrrolidine are stereoisomers. More preferably, the 1,2-epoxy-3N protected amine has a protected hydroxy methyl group. Further preferably, the 1,2-epoxy 3-N-protected amine has an aliphatic or aromatic group containing 1 to 10 carbon atoms, which can be unsaturated, branched or straight chain or cyclic. Still further, the reaction is in dimethylsulfoxide as the solvent at 80-85° C. for at least 24 hours. Further still, the amine is pre-formed by a base rearrangement of a 2,3-aziridin-1-ol. Further, the amine is pre-formed by a base rearrangement of a 2,3-aziridin-1-ol and wherein the base is the dimethylsulfoxonium methylide. Still further, the amine is pre-formed by a base rearrangement of a 2,3-aziridin-1-ol, wherein the base is the dimethylsulfoxonium methylide and wherein the process is performed sequentially in a single reactor. Further still, the amine is subsequently deprotected.

The present invention relates to a process for producing a 1,2-allylic 3- or 4-N protected amine which comprises reacting a 1,2-allylic 3-N protected aziridine with dimethylsulfonium methylide to produce the 1,2-allylic 3- or 4-N-protected amine. Preferably, the allylic 1,2-amine is converted to an amino acid by conversion of the 1,2-allylic group to a carboxylic acid group. More preferably, the 1,2-allylic 4-N-protected amine is converted to a diene and then cyclized to produce a piperidine.

### BRIEF DESCRIPTION OF THE DRAWINGS

FIG. 1 is a schematic drawing showing the formation of various drug compounds as examples of the utility of the present invention.

FIG. 2 is a drawing showing alternatives for the dimethylsulfoxonium methylide reactions. The C-1 reaction is favored.

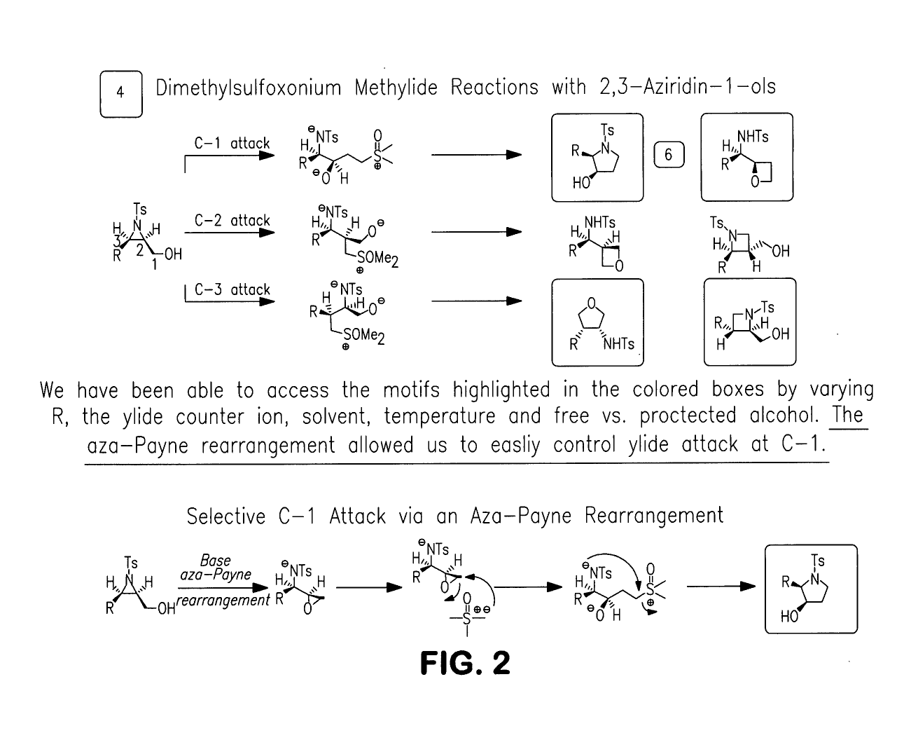

### DESCRIPTION OF PREFERRED EMBODIMENTS

In contrast to the Payne rearrangement, the aza-Payne rearrangement of activated 2,3-aziridin-1-ols (Scheme 2) has not received as much attention, despite its great potential for the synthesis of enantiomerically pure nitrogen-containing compounds.¹⁷ Ibuka and co-workers have described the aza-Payne rearrangement of a series of cis and trans-2,3-disubstituted aziridin-1-ols,

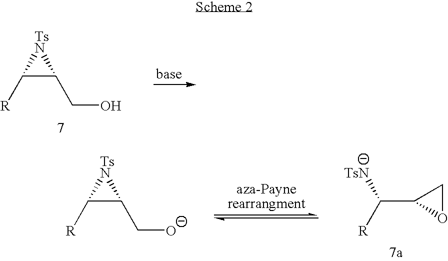

as well as the reaction of the resulting epoxy amines with a few selected nucleophiles, including organocuprates and amines.¹⁷⁻¹⁹ A particularly useful feature of the aza-Payne rearrangement is that under aprotic conditions, the equilibrium for both cis and trans-disubstituted 2,3-aziridin-1-ols lies exclusively towards the epoxy amine. This may result from the greater ability of the activated amine to stabilize the negative charge under the basic reaction conditions and/or the greater thermodynamic stability of the epoxy amine vs. the aziridinol. Thus, it was envisaged that an ylide-based aza-Payne rearrangement of 2,3-aziridin-1-ols could lead to an efficient process for the preparation of pyrrolidines (Scheme 3).^{16b}. The aza-Payne rearrangement is expected to favor epoxide 7a over aziridine 7, and subsequent nucleophilic attack to yield the bis-anion is anticipated. A 5-exo-tet ring closure of the bis-anion would yield the desired pyrrolidine 7b, thus completing the

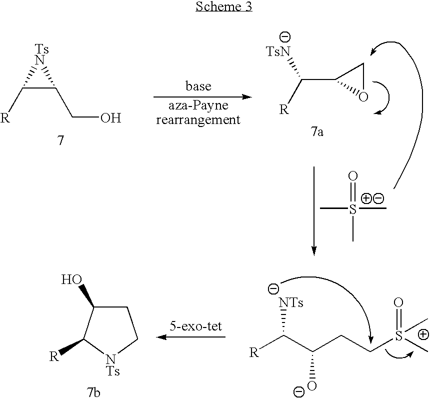

relay of the 3-membered to the 5-membered nitrogen containing ring system.

TABLE 1
Aza-Payne rearrangement of 2,3-aziridin-1-ols to epoxy amines

| entry | aziridinol | method | epoxy amine | yield |
|---|---|---|---|---|
| 1 | 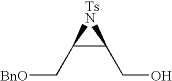 8 | A | 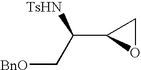 8a | 90% |
| 2 | 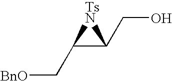 9 | A | 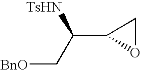 9a | 89% |
| 3 | 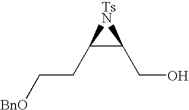 10 | A | 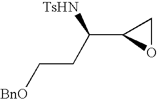 10a | 86% |
| 4 | 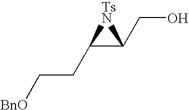 11 | AB | 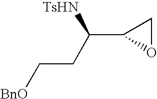 11a | 87%86% |
| 5 | 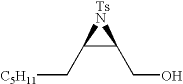 12 | A | 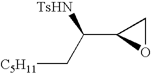 12a | 84% |
| 6 | 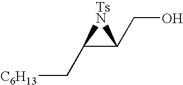 13 | A | 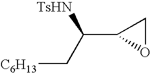 13a | 86% |
| 7 | 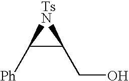 14 | A | 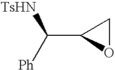 14a | 85% |
| 8 | 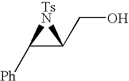 15 | AB | 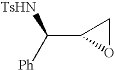 15a | 79%83% |
| 9 | 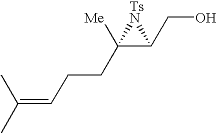 16 | A | 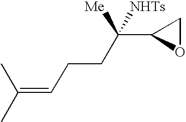 16a | 89% |
| 10 | 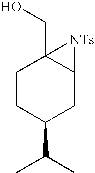 17 | A | 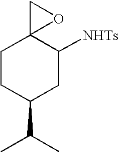 17a | 78% |
| 11 | 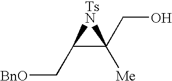 18 | A | 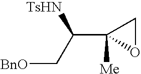 18a | 94% |
| 12 | 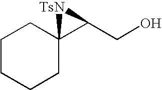 19 | A | 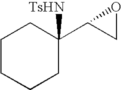 19a | 93% |
| 131415 | 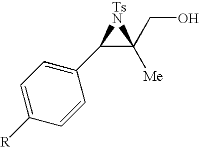 20, R = H21, R = OCH₃22, R = CF₃ | AAA | 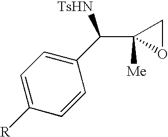 20a,21a,22a, | 81%93%66% |
| 16 | 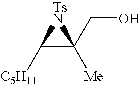 23 | A | 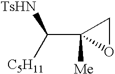 23a | 70% |
| 17 | 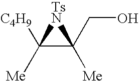 24 | C | 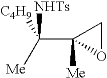 24a | 67% |
| 18 | 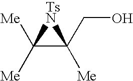 25 | C | 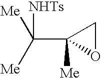 25a | 74% |

Method A: A 0.1 M solution of the aziridinol was treated with 4.0 equiv NaH in THF at rt;
Method B: A 0.1 M solution of the aziridinol in DMSO was treated with 4-8 equiv of dimethylsulfoxonium methylide at rt;
Method C: A 0.1 M solution of the aziridinol was treated with 4.0 equiv NaH in 20:1 to 12:1 THF/HMPA at rt.

### Aza-Payne Rearrangement

### EXAMPLES 1 TO 18

To our knowledge, the facility of the aza-Payne in more highly substituted compounds such as 2,3,3-, 2,2,3-trisubstituted and tetrasubstituted aziridinols, has not been well-studied. In order to utilize aziridinols for the synthesis of pyrrolidines, a closer inspection of the aza-Payne rearrangement for a variety of substituted aziridines was necessary. The desired aziridinols could be accessed in several ways. Enantiomerically pure 2,3-disubstituted aziridin-1-ols such as 8 and 9 (Table 1) could be obtained via the asymmetric epoxides 1 and 4 using literature procedures.^{21,22} Ring-opening of the corresponding epoxide with sodium azide was followed by a one-pot Staudinger reduction/cyclization and tosyl protection to give the desired aziridine. The Sharpless asymmetric aminohydroxylation could be used to synthesize disubstituted aziridinols.²³ The use of a VAPOL-catalyzed aziridination developed by the Wulff group could be implemented to access cis aryl-substituted compounds 24 Tetrasubstituted aziridinols could be accessed via stereoselective nucleophilic attack of an azirine as described by Davis and co-workers.²⁵ The majority of the racemic substrates were synthesized via treatment of the corresponding allylic alcohols with Chloramine T and catalytic NBS.²⁶

The aza-Payne rearrangement of tosylated aziridinols was accomplished in excellent yields using 4.0 equiv of NaH in THF or in some cases, dimethylsulfoxonium methylide in DMSO (Table 1). Other bases and solvents that could be utilized for the aza-Payne rearrangement include NaH in toluene, DMSO or 12:1 THF/HMPA, as well as KH in either THF or toluene. The use of NaHMDS or KHMDS in THF, toluene or DMSO was much less successful. In previous work directed towards the synthesis of tetrahydrofurans (Scheme 1), compounds containing an oxygen substituent at either C-4 or C-5 of the epoxy alcohol were excellent substrates for the Payne rearrangement/one carbon homologative ring-opening/cyclization sequence to yield 2,3-disubstituted tetrahydrofurans. Likewise, these substrates (entries 1-4, Table 1) performed well in the aza-Payne rearrangement by treatment with NaH in THF as described by Ibuka.¹⁷ There were no notable disparities in the yields of epoxy amines utilizing either cis or trans disubstitituted aziridinols as substrates (Table 1, entries 1-8). Alkyl epoxides analogous to 12 and 13 (entries 5-6) were not good substrates for Payne rearrangement but the corresponding 2,3-aziridin-1-ols 12 and 13 gave only epoxy amine products 12a and 13a with no trace of the starting materials. It was also of note that the 2,3,3-trisubstituted aziridinol 16 (entry 9) derived from geraniol was converted successfully to the epoxy amine 16a in excellent yield despite the fact the analogous geraniol epoxide was resistant to the Payne rearrangement and gave only 14% of the tetrahydrofuran product.^{16a} A cyclic 2,3,3-trisubstituted substrate 19 (entry 12) was also successful in the aza-Payne rearrangement, the analogous epoxy alcohol substrate giving only 21% yield of the desired product in our previous work.^{16a} Gratifyingly, 2,2,3-trisubstituted aziridinols such 17 and 18 (entries 10 and 11) underwent facile rearrangement even though the electrophilic center was tertiary to yield epoxy amines 17a and 18a, respectively. Relief of steric strain in the epoxide analogue of 17 in going from the fused ring system to the spiro ring system has been cited as the reason for the 1:2 mixture of 1,2-epoxy-3-ol and 2,3-epoxy-1-ol in the Payne rearrangement.²⁷ In contrast, aziridinol 17 leads to the production of only one isomer. A series of 2,2,3-trisubstituted aziridinols bearing an aryl group at C-3 were also examined. An electronic component to the facility of the aza-Payne rearrangement was noted, as the p-methoxyphenyl containing 21 gave 93% of the epoxy amine, while the trifluoromethylphenyl-containing substrate 22 gave only a 66% yield of 22a. The 2,2,3-trialkylsubstituted aziridinol 23 gave the epoxy amine 23a. Finally, the tetrasubstituted aziridinols 24 and 25 (entries 17-18) also underwent successful aza-Payne rearrangement to yield epoxy amines 24a and 25a. A small amount of HMPA was necessary to improve the yield of the transformation.¹⁷ The ability to use tri- and tetrasubstituted aziridinols in the aza-Payne rearrangement allows transfer of the terminal oxygen to a sterically congested tertiary center, yielding a quaternary hydroxyl center upon opening of the unhindered epoxide with various nucleophiles. This can yield synthetically useful 1,2-amino alcohols of various substitution patterns that might not otherwise be easily accessible.

Pyrrolidines from Aza-Payne Rearranged Aziridinols

### EXAMPLES 1 TO 12

The data in Table 1 illustrate the efficiency of the aza-Payne rearrangement for a number of aziridinols. In all cases the epoxide is isolated in high yields, however, more importantly, the rearrangement was facile using dimethylsulfoxonium methylide as the base. This is critical for the implementation of the next series of transformations. As depicted in Scheme 3, it was envisaged that epoxide 7a, obtained via the aza-Payne rearrangement of 7, could undergo nucleophilic trapping with the sulfoxonium ylide to yield the bis-anion intermediate. Ring closure with loss of dimethylsulfoxide would yield the desired pyrrolidine 7b.

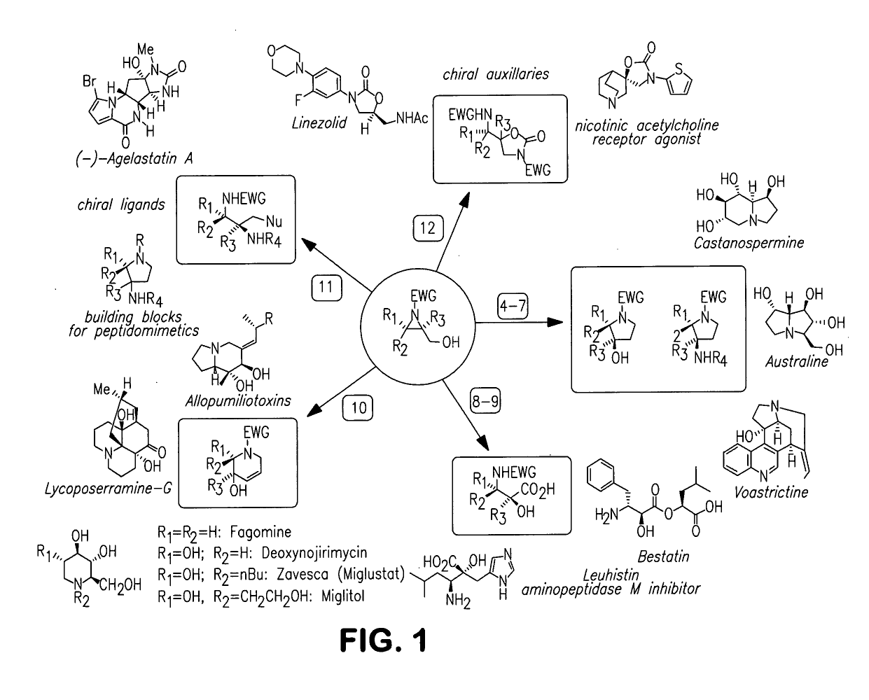

The epoxy amines that were generated in Table 1 were treated with dimethylsulfoxonium methylide in DMSO at 85° C. to afford the 2,3-disubstituted pyrrolidine rings in good to excellent yields and with complete control of diastereoselectivity (Table 2). Epoxy amine 8a generated from the cis aziridinol 8 led to the corresponding cis-disubstituted pyrrolidine ring 8b, while the epoxy amine 9a obtained from the trans aziridinol 9 gave the trans-disubstituted pyrrolidine 9b in excellent yields (Table 2, entries 1-2). The relative stereochemistries were verified by nOe experiments of the cis and trans pyrrolidines 8b and 9b that showed a greater enhancement of the H-2 proton when H-3 of the cis compound was irradiated as compared to the trans product. To further establish the relative stereochemistry of the substituents at C-2 and C-3, an X-ray crystal structure of compound 9b was obtained that clearly indicated the trans orientation of the C-2 and C-3 substituents (FIG. 1).

The remaining epoxy amines obtained from

TABLE 2
Conversion of epoxy amines to 2,3-substituted pyrrolidines.

| entry | epoxy amine | pyrrolidine | yield |
|---|---|---|---|
| 1 | 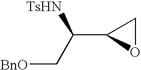 8a | 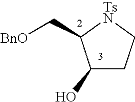 8b | 99% |
| 2 | 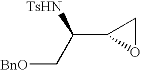 9a | 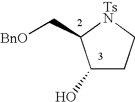 9b | 97% |
| 3 | 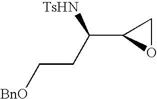 10a | 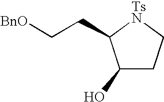 10b | 89% |
| 4 | 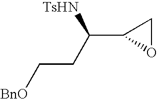 11a | 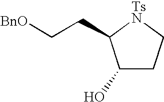 11b | 85% |
| 5 | 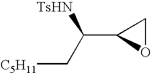 12a | 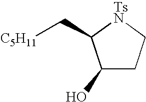 12b | 92% |
| 6 | 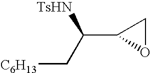 13a | 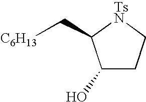 13b | 95% |
| 7 | 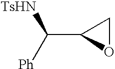 14a | 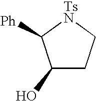 14b | 88% |
| 8 | 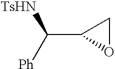 15a | 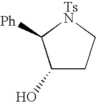 15b | 71% |
| 9 | 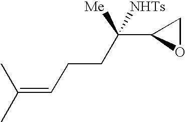 16a | 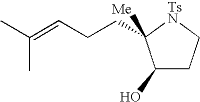 16b | 86% |
| 10 | 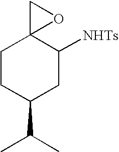 17a | 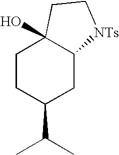 17b | 76% |
| 11 | 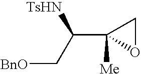 18a | 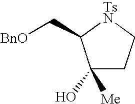 18b | 88% |
| 12 | 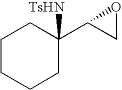 19a | 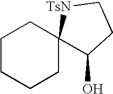 19b | 52% |

Reactions were run in as a 0.1 M solution of epoxy amine in DMSO using 4-8 equiv dimethylsulfoxonium methylide at 80-85° C. for 24 h.

disubstituted aziridinol substrates (entries 3-8) gave the corresponding pyrrolidines in high yields as single diastereoisomers. Epoxy amines derived from 2,3,3-trisubstituted aziridinols (entries 9 and 12, Table 2) also gave good yields of the pyrrolidines bearing a quaternary nitrogen center. This is again in contrast to the analogous reactions with similarly substituted epoxy alcohols as substrates, which gave the THF products in yields less than 30%. The steric hindrance of the
nucleophilic nitrogen of 19a in entry 12, along with the strain of the spiro ring system presumably led to a lower yield of pyrrolidine 19b. Entries 10 and 11 illustrate the conversion of epoxy amines 17a and 18a derived from 2,2,3-trisubstituted aziridinols to pyrrolidines that contain a chiral 3 hydroxyl group at C3. The relative stereochemistry of the trisubstituted pyrrolidine 18b was established via nOe enhancements observed between the benzyl protected hydroxymethyl side chain at C-2 and the C-3 methyl group. This again verified the anticipated stereochemical outcome of the reaction based on the mechanism depicted in Scheme 3.

### One-Pot Conversion of Aziridinols to Pyrrolidines

### EXAMPLES 1 TO 13

Having established high efficiency in the preparation of pyrrolidines from aziridinols in two steps, i.e.; aza-Payne rearrangement of aziridinols, followed by treatment of products isolated from the latter reaction with dimethylsulfoxonium methylide, our attention was directed towards a one-pot preparation of pyrrolidines. As such, the ylide itself would serve as the base to promote aza-Payne rearrangement (already shown to be effective, Table 1), leading to an epoxide that would undergo attack with the ylide (Scheme 3). Ring closure to the pyrrolidine would deliver the desired product. An issue of particular concern was competing ring-opening of the aziridine at either C-2 or C-3 by the ylide prior to aza-Payne rearrangement, particularly since excess ylide is used to compensate for its degradation at the elevated reaction temperatures. Several products could then be obtained depending on the relative nucleophilicities of the oxygen and nitrogen anions to form oxetanes, azetidines, tetrahydrofurans, or pyrrolidines (Scheme 4).²⁸

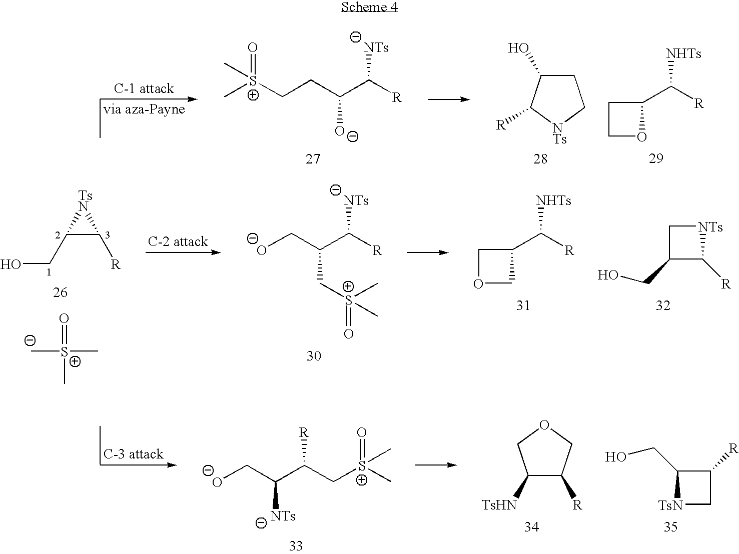

In order to determine the facility of epoxide vs. aziridine ring-opening with dimethylsulfoxonium methylide, a competition experiment was performed (Scheme 5). A 1:1 mixture of 36 and 38 was treated with 1.0 equiv of dimethylsulfoxonium methylide in DMSO at rt for 1 h, then 85° C. for 24 h. Epoxide 36 was recovered in 95% yield, while aziridine 38 was consumed completely. The azetidine 39 was obtained in 64% yield. Clearly, the higher reactivity of the aziridine as compared to the epoxide towards

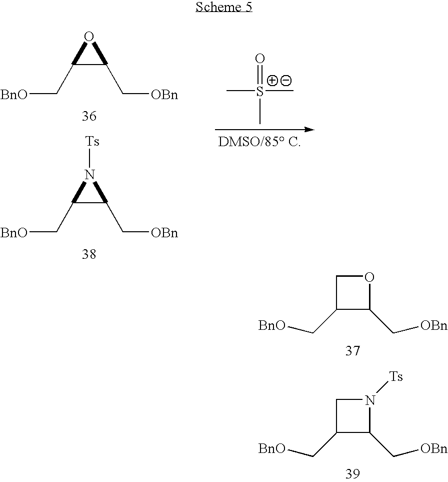

nucleophilic attack by the ylide could lead to the aforementioned mixture of undesired products (Scheme 4). However, two factors were crucial in our belief that a choreographed sequence of events (aza-Payne; nucleophilic attack of epoxide; ring closure) could be achieved (Scheme 3). First, the aza-Payne rearrangement is an intramolecular process and may compete favorably with the intermolecular process of aziridine ring opening with the ylide. Second, the epoxide that undergoes attack by the ylide is less hindered than the starting aziridine. Another important piece of information obtained from the experiment shown in Scheme 5 was that heating to 85° C. was necessary to cause ylide ring-opening of the aziridine. We reasoned that the aza-Payne rearrangement could be accomplished at a lower temperature, then the temperature raised to 85° C. to promote epoxide ring-opening and subsequent ring closure to the pyrrolidine. In this manner, the undesired ring-opening of the aziridine should not compete with the desired epoxide ring-opening by the ylide.

In the first successful attempt at a one-pot reaction to form pyrrolidines, the ylide generated from trimethylsulfoxonium iodide and NaH was added to the aziridinol, stirred at rt for 30 min, and heated to 85° C. for 24 h. Conversion of aziridinols 8 and 9 to pyrrolidines 8b and 9b occurred with moderate yields of 67% and 61%, respectively. We suspected that the lower yields might be caused by competitive ring-opening of the aziridine at the elevated reaction temperatures prior to complete aza-Payne rearrangement.

The effect of the ylide counterion on the efficacy of the aza-Payne/ring-opening/ring closure reaction was briefly examined. Dimethylsulfoxonium methylide was generated using both NaH and KH as bases in DMSO. Varying amounts of the ylide were added to solutions of 8 in DMSO and the reactions were stirred for 4 h at rt to ensure aza-Payne rearrangement was complete. After an additional 4 h, the reactions were analyzed by HPLC. The reactions utilizing potassium as the counterion were re-analyzed at 22 h. The results indicate the sodium counterion is more effective for ring closure to the desired pyrrolidine 8b, with a 90% conversion to product after 8 h. The potassium cation was less effective, resulting in a 40% conversion to 8b after 8 h. However, high conversions were obtained using KH by running the reaction overnight. As expected, increasing the amount of ylide from 2 to 10 equivalents greatly increased the rate, although this effect leveled out after 8 equivalents.

With these results in hand, the one-pot reaction was repeated with 8 using 8.0 equiv of ylide generated from NaH as the base in DMSO. The reaction was stirred at rt overnight and pyrrolidine 8b was obtained in 82% yield. However, the reaction was often not complete for trans and more hindered aziridinols, resulting in a mixture of pyrrolidine, epoxy amine and N-methylated products. Prolonged heating of the more stubborn reactions proved to be a general solution, and thus heating of all reactions at 80-85° C. for 24 h was adopted as standard protocol. These reactions could also be performed in THF with a small amount of DMSO (5 equiv). Substrate 8 (Table 3) was treated with dimethylsulfoxonium methylide (generated by refluxing trimethylsulfoxonium iodide with NaH in THF for 4 h), stirred at rt for 4 h and heated to 80° C. overnight in a sealed tube. In this manner, the pyrrolidine 8b could be obtained in 82% yield.

The general reaction conditions discussed above were adopted for the one-pot conversion of 2,3-aziridin-1-ols to pyrrolidines (Table 3). The cis-substituted aziridinols tended to give slightly higher yields than the corresponding trans analogues. As previously described in the discussion of Table 2, substrates containing alkyl substituents (entries 5-6) or trisubstituted aziridines (entries 9-12) proceeded under the reaction conditions to give pyrrolidines in good yields, in contrast to our analogous work using 2,3-epoxy-1-ols to prepare THFs. Aryl aziridines 14 and 15 (entries 7 and 8) were also successfully converted to pyrrolidines 14b and 15b, respectively, without any indication of C-3 ring-opening by the ylide. The major by-product in the trans aziridine substituted with an aryl group at C-3 (entry 10) was N-methylated epoxy amine. Other trans aziridinols were also prone to N-methylation if the temperature was lowered below 75° C. or any extra trimethylsulfoxonium iodide was present. The ee's of selected aziridinols and pyrrolidines were determined by preparing the corresponding MPA esters to ensure that racemization had not occurred under the reaction conditions (entries 1, 2, 7 and 9).²¹ Thus, a successful one-pot strategy for the synthesis of pyrrolidines from 2,3-aziridin-1-ols was developed by decoupling the roles of the ylide as a base and a nucleophile by judicious modulation of temperature.

TABLE 3
One-pot conversion of 2,3-aziridin-1-ols to 2,3-substituted pyrrolidines.

| entry | aziridinol | pyrrolidine | yield |
|---|---|---|---|
| 1 | 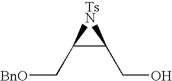 888% ee | 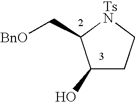 8b87% ee | 82% |
| 2 | 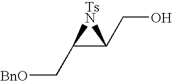 997% ee | 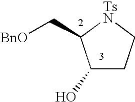 9b95% ee | 78% |
| 3 | 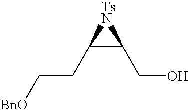 10 | 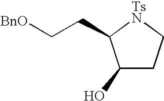 10b | 77% |
| 4 | 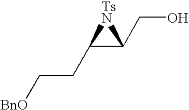 11 | 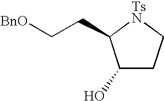 11b | 71% |
| 5 | 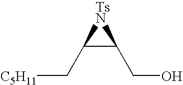 12 | 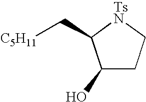 12b | 79% |
| 6 | 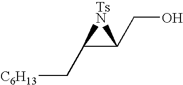 13 | 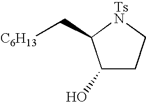 13b | 71% |
| 7 | 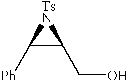 1499% ee | 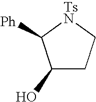 14b95% ee | 79% |
| 8 | 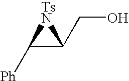 15 | 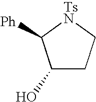 15b | 68% |
| 9 | 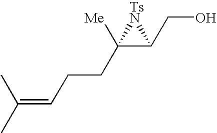 1667% ee | 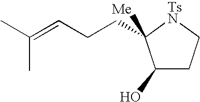 16b66% ee | 70% |
| 10 | 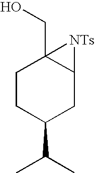 17 | 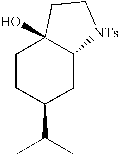 17b | 76% |
| 11 | 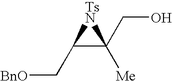 18 | 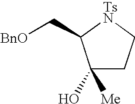 18b | 79% |
| 12 | 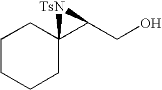 19 | 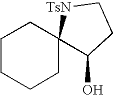 19b | 69% |
| 13 | 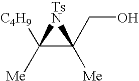 24 | 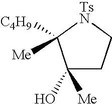 24b | 67% |

Reactions were run by stirring a 0.1 M solution of the aziridinol with excess dimethylsulfoxonium methylide in DMSO for 4 h, followed by heating at 80° C. for 24 h.

Lengthy reaction times for some of the sterically demanding aziridinols prompted a quick screen of possible remedies. Microwave reactions have the potential to decrease reaction times dramatically while increasing the yield.²⁹ Aziridinols 8, 13, and 16 were subjected to microwave irradiation studies to ascertain the potential reduction in reaction time for the preparation of pyrrolidines. The ylide was prepared as usual and the aziridinols were allowed to stir initially at rt for 4 h to ensure that aza-Payne rearrangement was complete. The reactions were then subjected to microwave irradiation (30 pulses, 15 sec/pulse). Gratifyingly, the reaction times of 8 and 13 decreased substantially (24 h vs. ˜8 min), and the yields of their corresponding products 8b and 13b increased to 91% (from 82%) and 79% (from 71%), respectively. Microwave-assisted reaction of aziridinol 16 produced a similar yield as compared to the typical conditions at a fraction of the time required with conventional heating. Further microwave studies are ongoing and the results will be reported in due course.

Having established the viability of generating substituted pyrrolidines in a stereocontrolled fashion, it was important to ensure that the activating groups could be removed efficiently to give the free pyrrolidine. The use of an electron-withdrawing group on the aziridine nitrogen was necessary to activate the ring towards nucleophilic ring opening but tosyl (Ts) groups can be difficult to remove under acidic or basic conditions. We wanted to examine other activating groups that allow for easier deprotection of the pyrrolidine products. Typical nitrogen protecting groups such as acetyl, Boc, and benzyl, did not facilitate aza-Payne rearrangement. The use of the tert-butylsulfonamide (Bus) group examples 1 and 2 as an activating/protecting group for aziridines has been documented and provides a complementary alternate to the Ts group as it can be easily removed under acidic conditions.³⁰ As depicted in Table 4, Bus analogues of aziridinols 8 and 15 (40 and 41 in Table 4) were synthesized and subjected to treatment with NaH in THF to effect the aza-Payne rearrangement. The yield of the aza-Payne rearrangement was lower as compared to the Ts-activated

TABLE 4
Use of a Bus protecting group.

| entry | aziridinol | epoxy amine (yield) | pyrrolidine | yield (one-pot)^{a} |
|---|---|---|---|---|
| 1 | 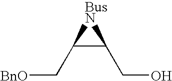 40 | 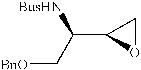 40a (70%) | 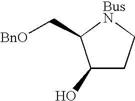 40b | 74% |
| 2 | 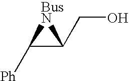 41 | 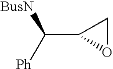 41a (61%) | 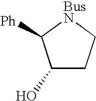 41b | 52% |

^{a}Epoxy amines were prepared by treating a 0.1 M solution of the aziridinol in THF with 4.0 equiv NaH and stirring at rt for 4-6 h.
^{b}The pyrrolidines were prepared by treating a 0.1 M solution of the aziridinol in DMSO with 4-8 equiv of dimethylsulfoxonium methylide, stirring at rt for 4 h and heating to 80° C. for 24 h.

counterpart, perhaps due to the sterics of the bulky t-butyl group or the decreased ability of the t-butylsulfonyl group to stabilize the resulting negative charge on nitrogen following rearrangement. The lower yield of pyrrolidine in the one-pot reaction to give 40b and 41b reflects the lower yield of the in situ aza-Payne rearrangement compared to the Ts-protected analogues. We also attempted the use of a —P(O)Ph₂ protecting group that has been documented to activate aziriaines towards nucleophilic ring opening, but can be removed under mild acidic conditions.³¹ However, we could not obtain aza-Payne rearranged products, much less the pyrrolidines.

### Removal of the Nitrogen Protecting Group

### EXAMPLES 1 TO 5

Finally, the pyrrolidine products were subjected to various deprotection conditions in order to ascertain the efficiency in removal of the nitrogen activating group (Table 5). The Ts protected pyrrolidines could be deprotected with sodium naphthalide in glyme in moderate to good yields, but a substantial amount of debenzylated product was also formed for benzyl protected substrates.³² In contrast, the Ts group was easily removed under mild conditions using Mg metal in MeOH (Table 5) to provide the free amines.³³ TBS-protected alcohols under the tosyl deprotection conditions were stable as can be seen in conversion of 42 to 42c. The p-methoxy variant of the Ts protecting group (see structure 43 in Table 5) was also utilized, however, it was less efficient. The Bus group in 40b was easily removed using conditions previously described by Weinreb

TABLE 5
Deprotection of pyrrolidine products.

| entry | substrate | product | yield |
|---|---|---|---|
| 1 | 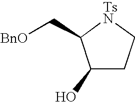 8b | 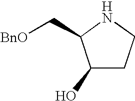 8c | 64%^{a} |
| 2 | 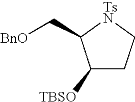 42 | 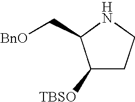 42c | 56%^{a} |
| 3 | 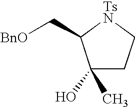 18b | 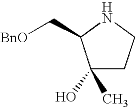 18c | 65%^{a} |
| 4 | 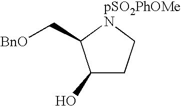 43 | 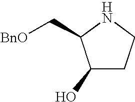 43c | 41% at78%conver-sion^{a} |
| 5 | 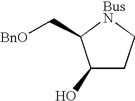 40b | 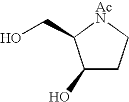 40c | 88%^{b} |

^{a}6.0 equiv Mg metal in MeOH, sonicate 30 min, rt overnight.
^{b}TfOH, p-anisole, CH₂Cl₂, −78° C., then NaOH/EtOAc.

(solution of triflic acid in MeOH with p-anisole as a cation scavenger) in good yields to provide the free amine.³⁴ The benzyl protecting group was also removed under these conditions, giving a highly polar product that was acetylated to facilitate isolation of 40c. Thus, we can remove the nitrogen protecting group under either acidic or basic conditions, provided care is taken in the protection of hydroxyls in the molecule.

### CONCLUSION

In conclusion, we have developed a new method for the synthesis of 2,3-disubstituted, 2,2,3- and 2,3,3-trisubstituted, and 2,2,3,3-tetrasubstituted pyrrolidine rings. The stereochemistry present in the asymmetric aziridinol is translated fully to the final product. Since it is simple to access these substrates in high enantiomeric excess via the Sharpless asymmetric epoxidation, Sharpless aminohydroxylation or Wulff VAPOL-catalyzed aziridination, this can be a powerful methodology for gaining entry into 2,3-substituted pyrrolidines with stereodefined substituents. Future work includes efforts to further functionalize the pyrrolidine ring via the use of aziridinols substituted at C-1 and utilization of substituted ylides.

General Procedures. Aza-Payne Rearrangement.

### Experimental Section

The aziridinol 8 (1.0 g, 2.9 mmol, 1.0 equiv) dissolved in a small amount of THF was added to a suspension of NaH (0.46 g as a 60% dispersion in mineral oil, 4.0 equiv, 11.5 mmol) in dry THF (30 mL). The reaction was stirred at rt for 4 h, then cooled to 0° C. and quenched carefully with saturated ammonium chloride. The aqueous layer was extracted 3× with portions of ethyl acetate and the combined organics were washed with brine. The organics were dried over sodium sulfate and the volatiles removed via rotary evaporation. The residue was purified by column chromatography (hexanes/ethyl acetate gradient) to give the epoxy amine 8a in 90% yield.

Synthesis of Pyrrolidines from Epoxy Amines.

A suspension of NaH (58.2 mg as a 60% dispersion in mineral oil (washed twice with dry pentane), 1.45 mmol, 5.0 equiv) in DMSO (3 mL, 0.1 M in aziridinol) was treated with trimethylsulfoxonium iodide (0.32 g, 1.45 mmol, 5.0 equiv) and the reaction stirred at rt for 30 min to give a milky-white solution. The epoxy amine 8a (0.1 g, 0.29 mmol, 1.0 equiv) dissolved in a small amount of DMSO was added to the ylide, stirred at rt for 30 min and heated to 80° C. for 24 h. The cooled reaction was quenched with saturated ammonium chloride (10 mL) and extracted 3× with portions of ethyl acetate. The combined organics were washed with brine, dried over sodium sulfate and the volatiles evaporated. The residue was purified by column chromatography (hexanes/ethyl acetate gradient) to give the desired pyrrolidine 8b in 99% yield as a thick oil that eventually crystallized to a low-melting solid.

Synthesis of Pyrrolidines from Aziridinols.

Dimethylsulfoxide was dried by stirring overnight over CaH₂ and distilled under high vacuum into a flame-dried flask containing activated molecular sieves. Trimethylsulfoxonium iodide was dried overnight at rt under high vacuum. Dimethylsulfoxonium methylide was prepared fresh for each reaction. Sodium hydride (0.32 g as a 60% dispersion in mineral oil, 8.0 mmol, 8.0 equiv, washed twice with pentane dried over sodium metal) was placed in a flame-dried flask and dry dimethylsulfoxide (10 mL) was added via syringe. Trimethylsulfoxonium iodide (1.77 g, 8.0 mmol, 8.0 equiv) was added in small portions over 20-30 min. After addition of the trimethylsulfoxonium iodide was complete, the reaction was stirred for an additional 30 min until the bubbling of the milk-white suspension ceased. The aziridinol 8 (0.35 g, 1.0 mmol, 1.0 equiv) dissolved in a small amount of DMSO was added dropwise and the reaction was stirred at rt for 4 h to complete the aza-Payne rearrangement. The reaction was then covered with aluminum foil and heated to 80-85° C. for 24 h. The dark brown mixture was cooled and diluted with 2× volume of water and saturated ammonium chloride (1 mL). The reaction was extracted several times with ethyl acetate, the combined organics were washed with brine and dried over sodium sulfate. After evaporation of the solvent, the residue was column chromatographed using a hexane/ethyl acetate gradient to give compound 8b in 82% yield as a thick oil that eventually crystallized to a low-melting solid.

Table of Content

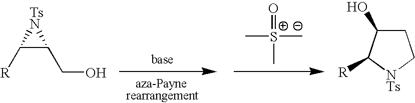

### REFERENCES

1. (1) a) Mroczek, T.; Glowniak, K. Proceedings of the Phytochemical Society of Europe 2002, 47, 1-46. b) Aurrecoechea, J. M.; Fernandez, A.; Gorgojo, J. M.; Saornil, C. Tetrahedron 1999, 55, 7345-7362 and references therein. c) Braekman, J. C.; Daloze, D. Studies in Natural Products Chemistry; Atta-ur-Rahman, Ed.; Elsevier: Amsterdam, 1990, 6, 421-466. d) Pichon, M.; Figadere, B. Tetrahedron: Asymmetry 1996, 7, 927-964. e) O'Hagan, D. Nat. Prod. Rep. 2000, 17, 435-446. f) O'Hagan, D. Nat. Prod. Rep. 1997, 14, 637-651. g) Elbein, A.; Molyneux, R. I. in Alkaloids: Chemical and Biological Perspectives; Pelletier, S. W., Ed.; John Wiley: New York, 1990; Vol. 5, pp. 1-54. h) Asano, N.; Nash, R. J.; Molyneux, R. J.; Fleet, G. W. Tetrahedron Asymm. 2000, 11, 1645-1680. i) Nishimura, Y. Japanese J. of Clin. Chem. 1993, 180-185. j) O'Hagan, D. Nat. Prod. Reports 2000, 17, 436-446. k) Patel, A. V.; Crabb, T. A. Pyrroles, pyrrolines and pyrrolidines. Rodd's Chemistry of Carbon Compounds; 1997; Vol. 4 (Part A), pp 457-556. l) Uchide, N.; Kunio, O. J. Antimicr. Chem. 2003, 52, 8-10. m) Wiedeman, P. E.; Trevillyan, J. M. Curr. Opin. Invest. Drugs 2003, 4, 412-420. n) Garcia-Morena, I. M.; Rodriguez-Lucena, D.; Ortiz-Mellet, C.; Garcia-Fernandez, J. M. Org. Lett. 2004, 6, 2003-2006. o) Kam, T.; Sim, K.; Lim, T. Tetrahedron Lett. 2001, 4721-4723. p) Ohtsu, Y.; Sasamura, H.; Tsurumi, Y.; Yoshimura, S.; Takase, S.; Hashimoto, M.; Shibata, T.; Hino, M.; Fujii, T. J. Antibiotics 2003, 56, 682-688.
(2) a) Huryn, D. M. Comprehensive Organic Synthesis; Trost, B. M.; Fleming, I.; Eds.; Pergamon: Oxford, 1991; Vol. 1, pp. 64-71. b) Enders, D.; Klatt, M. Synthesis 1996, 1403-1418. c) Yamamoto, Y.; Hoshino, J.; Fujimoto, Y.; Ohmoto, J.; Sawadi, S. Synthesis 1993, 298-302. d) Koh, K.; Ben, R. N.; Durst, T. Tetrahedron Lett. 1994, 35, 375-378. e) Corey, E. J.; Yuen, P.-W.; Hannon, F. J.; Wierda, D. A. J. Org. Chem. 1990, 55, 784-786. f) DeNinno, M. P.; Perner, R. J.; Lijewski, L. Tetrahedron Lett. 1990, 31, 7415-7418. g) Jones, T. J.; Mohan, J. J.; Xavier, L. C.; Blacklock, T. J.; Mathre, D. J.; Sohar, P.; Jones, E. T.; Reamer, R. A.; Roberts, F. E.; Grabowski, E. J. J. Org. Chem. 1991, 56, 763-769.
(3) a) Porter, E. A.; Wang, X.; Schmitt, M. A.; Gellman, S. H. Org. Lett. 2002, 4, 3317-3319. b) Porter, E. A.; Weisblum, B.; Gellman, S. H. J. Am. Chem. Soc. 2002, 124, 7324-7330.
(4) a) Pearson, W. H.; Clark, R. B. Tetrahedron Lett. 1997, 38, 7669-7672. b) Bashiardes, G.; Safir, I.; Mohamed, A. S.; Barbot, F.; Laduranty, J. Org. Lett. 2003, 5, 4915-4918. c) Bashiardes, G.; Safir, I.; Barbot, F.; Laduranty, J. Tetrahedron Lett. 2003, 44, 8417-8420. d) Galliford, C. V.; Beenen, M. A.; Nguyen, S. T.; Scheidt, K. A. Org. Lett. 2003, 5, 3487-3490. e) Young, I. S.; Williams, J., L.; Kerr, M. A. Org. Lett. 2005, 7, 953-955. f) Pearson, W. H.; Dietz, A.; Stoy, P. Org. Lett. 2004, 6, 1005-1008.
(5) a) Boto, A.; Hernandez, R.; de Leon, Y.; Suarez, E. J. Org. Chem. 2001, 66, 7796-7803. b) Boto, A.; Hernandez, R.; Suarez, E. Tetrahedron Lett. 2000, 41, 2495-2498.
(6) a) Nakhla, J. S.; Kampf, J. W.; Wolfe, J. P. J. Am. Chem. Soc. 2006, 128, 2893-2901. b) Ney, J. E.; Hay, M. B.; Yang, Q.; Wolfe, J. P. Adv. Synth. Catal. 2005, 347, 1614-1620. d) Beaudoin Bertrand, M.; Wolfe, J. P. Tetrahedron 2005, 61, 6447-6459. d) Yang, Q.; Ney, J. E.; Wolfe, J. P. Org. Lett. 2005, 7, 2575-2578. e) Ney, J. E.; Wolfe, J. P. Ang. Chem., Int. Ed. 2004, 43, 3605-3608.
(7) a) Wang, Q.; Sasaki, N. A.; Potier, P. Tetrahedron Lett. 1998, 39, 5755-5758. b) Sasaki, N. A.; Hashimoto, C.; Potier, P. Tetrahedron Lett. 1987, 28, 6069-6072. c) Sasaki, N. A.; Pauly, R.; Fontaine, C.; Chiaroni, A.; Riche, C.; Potier, P. Tetrahedron Lett. 1994, 35, 241-244. d) Sasaki, N. A.; Dockner, M.; Chiaroni, A.; Riche, C.; Potier, P. J. Org. Chem. 1997, 62, 765-770. e) Sasaki, N. A.,; Sagnard, I. Tetrahedron 1994, 50, 7093-7108. f) Dockner, M.; Sasaki, N. A.; Potier, P. Heterocycles 1996, 42, 529-532. g) Dockner, M.; Sasaki, N. A.; Riche, C.; Potier, P. Liebigs. Ann./Recueil 1997, 1267-1272. h) Back, T. G.; Parvez, M.; Zhai, H. J. Org. Chem. 2003, 68, 9389-9393.
(8) a) Miura, K.; Hondo, T.; Nakagawa, T.; Takahashi, T.; Hosomi, A. Org. Lett. 2000, 2, 385-388 and references therein.
(9) Coldham, I.; Hufton, R.; Price, K. N.; Rathmell, R. E.; Snowden, D. J.; Vennall, G. P. Synthesis 2001, 10, 1523-1531.
(10) a) Besev, M.; Engman, L. Org. Lett. 2002, 4, 3023-3025. b) Aurrecoechea, J. M.; Fernandez, A.; Gorgojo, J. M.; Saornil, C. Tetrahedron 1999, 55, 7345-7362 and references therein.
(11) Schlummer, B.; Hartwig, J. F. Org. Lett. 2002, 4, 1471-1474.
(12) a) Sletten, E. M.; Liotta, L. J. J. Org. Chem. 2006, 71, 1335-1343. b) De Raadt, A.; Ekhart, C. W.; Ebner, M.; Stutz, A. E. Topics Curr. Chem. 1997, 187, 157-186. c) Buchanan, J. G.; Edgar, A. R.; Hewitt, B. D.; Jigajinni, V. B.; Singh, G.; Wightman, R. H. in ACS Symposium Series 1989, 386, 107-116. d) Zhao, H.; Cheng, S.; Mootoo, D. R. J. Org. Chem. 2001, 66, 1761-1767.
(13) a) Ohno, H.; Takeoka, Y.; Kadoh, Y.; Miyamura, K.; Tanaka, T. J. Org. Chem. 2004, 69, 4541-4544. b) Apte, S.; Radetich, B.; Shin, S.; RajanBabu, T. V. Org. Lett. 2004, 6, 4053-4056.
(14) a) Pyne, S. G.; Davis, A. S.; Gates, N. J.; Hartley, J. P.; Lindsay, K. B.; Machan, T.; Tang, M. Synlett 2004, 15, 2670-2680. b) Felpin, F.; Lebreton, J. Eur. J. Org. Chem. 2003, 3693-3712. c) Cren, S.; Wilson, C.; Thomas, N. R. Org. Lett. 2005, 7, 3521-3523.
(15) Payne, G. B. J. Org. Chem. 1962, 27, 3819-3822.
(16) a) Schomaker, J. M.; Reddy, P. V.; Borhan, B. J. Am. Chem. Soc. 2004, 126, 13600-13601. b) During the submission of this manuscript we were made aware of a recent disclosure that bears some similarity to this work, although clearly both our groups have pursued their efforts independently. In the following manuscript the authors report the conversion of epoxy amines to pyrrolidines with the use of sulfoxonium ylides. Hodgson, D. M.; Fleming, M. J.; Xu, Z.; Lin, C.; Stanway, S. J. Chem. Commun. 2006, 3226-3228.
(17) a) Ibuka, T. Chem. Soc. Rev., 1998, 27, 145-154. b) Ibuka, T.; Nakai, K.; Akaji, M.; Tamamura, H.; Fujii, N.; Yamamoto, Y. Tetrahedron, 1996, 52, 11739-11752. c) Nakai, K.; Ibuka, T.; Otaka, A.; Tamamura, H.; Fujii, N.; Yamamoto, Y. Tetrahedron Lett. 1995, 36, 6247-6250. d) Ibuka, T.; Nakai, K.; Habashita, H.; Hotta, Y.; Otaka, A.; Tamamura, H.; Fujii, N.; Mimura, N.; Yoshihisa, M. J. Org. Chem., 1995, 60, 2044-2058. e) Najime, R.; Pilard, S., Vaultier, M. Tetrahedron Lett., 1992, 33, 5351-5354. f) Bouyacoub, A.; Volatron, F. Eur. J. Org. Chem. 2002, 24, 4143-4150. g) Rosser, C. M.; Coote, S. C.; Kirby, J. P.; O'Brien, P.; Caine, D. Org. Lett. 2004, 6, 4817-4819. h) Dollt, H.; Zabel, V. Australian J. Chem. 1999, 52, 259-270. i) Moulines, J.; Charpentier, P.; Bats, J.-P.; Nuhrich, A.; Lamidey, A.-M. Tetrahedron Lett. 1992, 33, 487. j) Atkinson, R. S.; Fawcett, J.; Russell, D. R.; Williams, P. J. Tetrahedron Lett. 1995, 36, 3241 and references cited therein.
(18) a) Fuji, K.; Kawabata, T.; Diryu, Y.; Suyiura, Y. Heterocycles 1996, 42, 701-722. b) Ibuka, T.; Nakai, K.; Habashita, H.; Fujii, N.; Garrido, F.; Mann, A. Tetrahedron Lett. 1993, 34, 7421-7424.
(19) a) Xu, Q.; Borremans, F.; Devreese, B. Tetrahedron Lett. 2001, 42, 7261-7263. b) Shi, T.; Rabenstein, D. L. Bioorg. Med. Chem. Lett. 2002, 12, 2237-2240.
(20) a) Nadir, U. K.; Sharma, R. L.; Koul, V. K. Tetrahedron 1989, 45, 1851-1858. b) Okuma, K.; Tanaka, Y.; Kaji, S.; Ohta, H. J. Org. Chem. 1983, 48, 5134-5135. c) Fitton, A. O.; Hill, J.; Jane, D. E.; Millar, R. Synthesis 1987, 1140-1142.
(21) Fujii, N.; Nakai, K.; Habashita, H.; Hotta, Y.; Tamamura, H.; Otaka, A.; Ibuka, T. Chem. Pharm. Bull. 1994, 42, 2241-2250.
(22) a) Nayak, S. K.; Thijs, L.; Zwanenburg, B. Tetrahedron Lett. 1999, 40, 981. b) Andres, J. M.; de Elana, N.; Pedrosa, R.; Perez-Encabo, A. Tetrahedron 1999, 55, 14137. c) Nayak, S. K.; Thijs, L.; Zwandenburg, B. Synlett 1998, 1187. d) Hwang, G. I.; Chung, J. H.; Lee, W. K. J. Org. Chem. 1996, 6183.
(23) a) Kolb, H. C.; Sharpless, B. K. Asymmetric aminohydroxylatich in Transition Metals for Organic Synthesis (2^{nd} edition), (2004), 2, 309-326. b) Schlingloff, G.; Sharpless, K. B. Asymmetric aminohydroxylation in Asymmetric Oxidation Reactions (2001), 104-114.
(24) a) Patwardhan, A. P.; Lu, Z.; Pulgam, V. R.; Wulff, W. D. Org. Lett. 2005, 7, 2201-2204. b) Antilla, J. C.; Wulff, W. D. Angew. Chem., Int. Ed. 2000, 39, 4518-4521. c) Antilla, J. C.; Wulff, W. D. J. Am; Chem. Soc. 1999, 121, 5099-5100. d) Patwardhan, A. P.; Pulgam, V. R.; Zhang, Y.; Wulff, W. D. Angew. Chem. Int. Ed. 2005, 44, 6169-6172.
(25) a) Davis, F. A; Ramachandar, T.; Wu, Y. J. Org. Chem. 2003, 68, 6894-6898. b) Davis, F. A.; Deng, J.; Zhang, Y.; Haltiwanger, R. C. Tetrahedron 2002, 58, 7135-7143. c) Davis, F. A.; Liu, H.; Liang, C.; Reddy, G. V.; Zhang, Y.; Fang, T.; Titus, D. D. J. Org. Chem. 1999, 64, 8929-8935. d) Davis, F. A.; Liang, C.; Liu, H. J. Org. Chem. 1997, 62, 3796-3797.
(26) Thakur, V. V.; Sudalai, A. Tetrahedron Lett. 2003, 44, 989-992.
(27) Swindell, C. S.; Britcher, S. F. J. Org. Chem. 1986, 51, 793-797.
(28) Gao, Y.; Klunder, J. M.; Hanson, R. M.; Masamune, H.; Ko, S. Y.; Sharpless, K. B. J. Am. Chem. Soc. 1987, 109, 5765-5780.
(29) Microwave Assisted Organic Synthesis Tierney, J. P.; Lidstrom, P.; ed.; Blackwell Publishing: Oxford, 2005.
(30) Gontcharov, A. V.; Liu, H.; Sharpless, K. B. Org. Lett. 1999, 1, 783-786.
(31) Sweeney, J. B.; Cantrill, A. A. Tetrahedron 2003, 79, 3677-3690.
(32) Alonso, D. A.; Andersson, P. G. J. Org. Chem. 1998, 63, 9455. b) Casadei, M. A.; Gessner, A.; Inesi, A.; Achille, J.; Werner, M.; Micheletti, F. J. Chem. Soc., Perk. Trans. I 1992, 122, 7-9.
(33) Pak, C. S.; Kim, T. H.; Ha, S. J. J. Org. Chem. 1998, 63, 10006-10010.
(34) Sun, P.; Weinreb, S. M. J. Org. Chem. 1997, 62, 8604.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

We claim:

1. A process for the preparation of a 2,3-di- or tri-substituted pyrrolidine which comprises reacting 1,2-epoxy-3-N protected amine with dimethylsulfoxonium methylide in an aprotic solvent to produce the 2,3-di- or tri-substituted pyrrolidine.

2. The process of claim 1 wherein the 1,2-epoxy-3-protected amine and the 2,3-pyrrolidine are stereoisomers.

3. The process of claim 1 wherein the 1,2-epoxy-3N protected amine has a protected hydroxy methyl group.

4. The process of claim 1 wherein the 1,2-epoxy 3-N-protected amine has an aliphatic or aromatic group containing 1 to 10 carbon atoms, which can be unsaturated, branched or straight chain or cyclic.

5. The process of any one of claims 1, 2, 3 or 4 wherein the reaction is in dimethylsulfoxide as the solvent at 80-85° C. for at least 24 hours.

6. The process of any one of claims 1, 2, 3 or 4 wherein the amine is pre-formed by a base rearrangement of a 2,3-aziridin-1-ol.

7. The process of any one of claims 1, 2, 3 or 4, wherein the amine is pre-formed by a base rearrangement of a 2,3-aziridin-1-ol and wherein the base is the dimethylsulfoxonium methylide.

8. The process of any one of claims 1, 2, 3 or 4 wherein the amine is pre-formed by a base rearrangement of a 2,3-aziridin-1-ol, wherein the base is the dimethylsulfoxonium methylide and wherein the process is performed sequentially in a single reactor.

9. The process of any one of claims 1, 2, 3 or 4 wherein the amine is subsequently deprotected.

10. A process for producing a 1,2-allylic 3- or 4-N protected amine which comprises reacting a 1,2-allylic 3-N protected aziridine with dimethylsulfonium methylide to produce the 1,2-allylic 3- or 4-N-protected amine.

11. The process of claim 10 wherein the allylic 1,2-amine is converted to an amino acid by conversion of the 1,2-allylic group to a carboxylic acid group.

12. The process of claim 10 wherein the 1,2-allylic 4-N-protected amine is converted to a diene and then cyclized to produce a piperidine.

13. The use of any one of compounds produced by the process of claims 1, 2, 3, 4, 5, 6, 7, 8, 9, 10, 11 or 12 or described in the attached description as an anti-cancer or anti-tumor or other pharmaceutical agent.
